(19) United States
(12) Patent Application Publication
(10) Pub. No.: US 2008/0200452 A1
(43) Pub. Date: Aug. 21, 2008

(54) Oral, Rapidly Disintegrating Film, Which Cannot be Spat Out, for a Neuroleptic

(76) Inventors: Petra Obermeier, Munchen (DE); Thomas Kohr, Munchen (DE); Kai-Thomas Kramer, Bad Tolz (DE); Karin Klokkers, Reichersbeveunl (DE)

Correspondence Address: LICATA & TYRRELL P.C., 66 E. MAIN STREET, MARLTON, NJ, 08053, US

(21) Appl. No.: 11/996,382

(22) PCT Filed: Jul. 20, 2006
(86) PCT No.: PCT/EP06/07177

§ 371 Date: Mar. 28, 2008

(30) Foreign Application Priority Data
Jul. 20, 2005 (DE) 10 2005 033 943.3

(51) Int. Cl. A61K 31/551; A61P 25/18

(52) U.S. Cl. 514/215

(57) ABSTRACT

Film-form, single-layered, cavity-free preparation free of surfactants, effervescent additive and taste masker and comprising one or more film former(s), one or more gel former(s) and one or more active ingredient(s) from the group of neuroleptics.

The invention relates to an oral, rapidly disintegrating, single-layered film, which cannot be spat out, comprising a neuroleptic, to its production and to its use. Preferably olanzapin is used as neuroleptic.

Pharmaceutical dosage forms, such as, for example, meltable tablets, which adhere to the mouth and rapidly disintegrate, are advantageous in a wide variety of respects. They facilitate oral administration of medicaments to patients suffering from psychic disorders, such as schizophrenia, who are difficult to treat with other oral medicament forms (e.g. film-coated tablets). By virtue of the mucoadhesiveness and rapid disintegration of the dosage form, the patient cannot keep the medicament form in, for example, the oral cavity and later spit it out again. A disadvantage of meltable tablets, however, is their cost-intensive production which requires an elaborate lyophilisation process; see, for example, DE 27 44 493, EP 0 793 495 and WO 01/39 836. Furthermore, some active ingredients, such as, for example, olanzapine, have only limited chemical stability in film-coated tablets.

As oral medicament forms that are mucoadhesive and rapidly disintegrate in the mouth there also come into consideration flat films. These are distinguished by a small layer thickness and accordingly by a large surface area, which brings about rapid disintegration.

For example, EP 936 905 describes mucoadhesive films comprising hypnotics, anti-epileptics or psychoneurotropics, which films contain a surfactant. A disadvantage of using surfactants, however, is their potential for causing irritation to the skin or mucosa. In addition, many of the customary surfactants have a very bitter taste. The possibility of interaction when the active ingredient is absorbed in the gastro-intestinal tract is likewise a disadvantage.

WO 03/101 420 describes films having a reduced tendency to adhere to the oral mucosa, and WO 03/070 227 describes mucoadhesive films, there being described in each case films, for example comprising psychopharmaceuticals, which contain a carbon dioxide former as effervescent additive. Disadvantages of an effervescent additive are its acidic taste and the formation of foam in the mouth. In addition, the formulation is very moisture-sensitive. The possibility of chemical interaction between the effervescent constituents and the adjuvants of the formulation is also disadvantageous.

WO 02/02085 discloses films having a reduced tendency to adhere to the oral mucosa and having cavities to reduce adhesion of the film to the oral mucosa.

WO 01/70194 and US 20040247649 describe mucoadhesive films comprising water-soluble polymers, a taste masker and active ingredients, for example psychopharmaceuticals.

The aim of the invention is to provide a film, which cannot be spat out, comprising a neuroleptic, especially olanzapine. The film should be suitable for oral administration of the neuroleptic. After making contact with liquid or saliva, the film should adhere to the mouth, where it should rapidly disintegrate, for example it should be dissolved or decomposed under the action of saliva. The active-ingredient-containing film should be both chemically and physically stable. The film should be free of the above-mentioned surfactants, effervescent additives or taste maskers. The film should be economical to produce.

To solve that problem the invention provides a preparation in film form which comprises one or more film former(s), one or more gel former(s) and one or more active ingredient(s) from the group of neuroleptics. The film-form preparation is preferably single-layered and preferably substantially free of cavities, surfactants, effervescent additives and taste maskers. Preferably, the film-form preparation is a film, especially a solid film. Preferably, the film is single-layered and comprises one or more film former(s), one or more gel former(s) and one or more active ingredient(s). Preferably, the film is substantially free of cavities, surfactants, effervescent additive and taste maskers. Preferably, the film disintegrates rapidly in saliva.

It has been found that the preparation according to the invention offers a very advantageous combination of mechanical stability of the film and rapid release of the active ingredient.

For example, an embodiment of the invention relates to a single-layered film-form preparation, comprising one or more film former(s), one or more gel former(s) and one or more active ingredient(s). Preferably, the film-form preparation is substantially free of cavities, surfactants, effervescent additive and taste maskers.

The expression “single-layered film-form preparation” preferably denotes a solid preparation which is in the form of a single-layered film, “single-layered” meaning that the film is in the form of a single layer, the layer preferably being homogeneous. The film can be flexible or non-flexible, but is preferably flexible.

Preferably, the single-layered film-form preparation is substantially free of cavities, a “cavity” being understood as being a region which is filled with a fluid (a gas and/or a liquid). Such a cavity usually has a diameter of less than 100 μm. Preferably, a film-form preparation is substantially free of gas bubbles and/or cavities that contain a fluid (gas and/or liquid).

Preferably, the single-layered film-form preparation is substantially free of surfactants, “substantially free of surfactants” meaning that the film-form preparation, based on the total preparation, contains less than 1% by weight, based on the dried preparation, preferably less than 0.1% by weight and especially less than 0.01% by weight surfactant. In particular, no surfactants are added as constituent during the production of the film-form preparation. A surfactant in the context of this invention is any customary surfactant, wetting agent or surface-active substance.

Preferably, the single-layered film-form preparation is substantially free of effervescent additive, “substantially free of effervescent additive” meaning that the film-form preparation, based on the total preparation, contains less than 1% by weight, based on the dried preparation, preferably less than 0.1% by weight and especially less than 0.01% by weight effervescent additive. In particular, no effervescent additive is added as constituent during the production of the film-form preparation. An effervescent additive in the context of this invention is a compound that releases a gaseous compound on addition of water, on storage, at elevated temperature or the like. Preferably, an effervescent additive is a compound that releases a gaseous compound in the mouth, for example under the action of saliva, such as, for example, a carbon dioxide former. The film-form preparation therefore contains no or almost no effervescent additive, such as, for example, a carbon dioxide former.

Preferably, the single-layered film-form preparation is substantially free of taste maskers, “substantially free of taste maskers” meaning that the film-form preparation, based on the total preparation, contains less than 1% by weight, based on the dried preparation, preferably less than 0.1% by weight and especially less than 0.01% by weight taste masker. In particular, no taste markers are added as constituent during the production of the film-form preparation. A taste masker in the context of this invention interacts with a substance having an unpleasant taste, with the result that the latter's unpleasant taste is “masked”.

A “taste masker” is to be understood as especially being a substance that serves to cover the unpleasant taste of, for example, an active ingredient. The film or the film-form preparation is, in particular, free of mixtures of the active ingredient with ion exchange resins, inclusion compounds of the active ingredient with cyclodextrin or coatings of the active ingredient with a covering, for example Eudragit. Preferably, the active ingredient is contained in the preparation in free form and is not, for example, encapsulated or enclosed.

A further embodiment relates to film-form, single-layered and preferably cavity-free preparations free of surfactants, effervescent additive and taste maskers and comprising one or more film former(s), one or more gel former(s) and one or more active ingredient(s) from the group of neuroleptics.

Olanzapine is preferred as neuroleptic for the preparation according to the invention.

The preparation according to the invention is free of taste maskers, but can optionally comprise sweeteners or flavourings.

In the preparation according to the invention, the active ingredient content in the film can be from 0.1 to 60% by weight and especially up to 50% by weight and preferably from 20 to 30% by weight and more especially about 25% by weight, in each case based on the dried preparation.

For the preparation according to the invention, one or more film former(s) from the following group can be provided:
sugar, sugar alcohols and derivatives thereof, especially saccharose, sorbitol, mannitol, xylitol, glucose, fructose, lactose and galactose,
low molecular weight organic acids, especially citric acid, succinic acid, malic acid and adipic acid,
polyethylene glycol, polyethylene glycol dioleate, 1,3-butanediol, propylene glycol, glycerol, isopropyl palmitate, dibutyl sebacate, paraffin oil and castor oil,
ethylcellulose,
cellulose acetate,
cellulose phthalate,
and mixtures of such film formers.

For the preparation according to the invention there are preferred one or more film former(s) from the group formed by sorbitol, xylitol, polyethylene glycol, polyethylene glycol dioleate, 1,3-butanediol, propylene glycol, isopropyl palmitate, dibutyl sebacate, paraffin oil, ethylcellulose, cellulose acetate and cellulose phthalate.

It is especially preferable for at least one film former to be insoluble in water. Especially preferred water-insoluble film formers are water-insoluble ethylcellulose, water-insoluble cellulose acetate and water-insoluble cellulose phthalate, and also paraffin oil.

According to the invention, “water-insoluble” is preferably defined as follows: 1 part of a compound (1 part film former or gel former) especially in accordance with the German Pharmacopoeia (9th edition of Jan. 1, 1987) is soluble in from 30 to 100 parts water, especially in from 100 to 1000 parts water, more especially in from 1000 to 10,000 parts water and very especially in more than 10,000 parts water. “Water-soluble” is preferably defined as follows: 1 part of a compound (1 part film former or gel former) especially in accordance with the German Pharmacopoeia (9th edition of Jan. 1, 1987) is soluble in from 10 to 30 parts water, especially in from 1 to 10 parts water and more especially in less than 1 part water.

In the preparation according to the invention, the film can contain film former in an amount of from 5 to 70% by weight, preferably from 5 to 30% by weight, in each case based on the dried preparation.

A film former in the context of this invention is especially a compound that imparts to the film preparation a certain degree of flexibility in terms of mechanical properties, such as, for example, resilience, flexural modulus, elasticity modulus and the like.

For the preparation according to the invention, at least one gel former from the following group can be provided:
polymeric carbohydrates, especially cellulose and derivatives thereof, preferably hydroxypropylcellulose (HPC), hydroxypropylmethylcellulose (HPMC), starch and derivatives thereof, agar-agar, alginic acid, arabinogalactan, galactomannan, carrageenan, dextran, tragacanth and gum of vegetable origin,
synthetic polymers that are soluble or swellable in water, especially polyvinylpyrrolidone, polyvinyl alcohol, polyacrylic acid and polyacrylamide,
polypeptides, especially gelatin, albumin and collagen, and
mixtures of such gel formers.

In the preparation according to the invention, the film can contain gel former in an amount of from 10 to 70% by weight, preferably from 20 to 50% by weight, in each case based on he dried preparation.

A gel former in the context of this invention is especially a polymeric compound having a molecular weight of less than 60,000 Dalton, preferably from 10,000 to 40,000 Dalton. Polymeric compounds of such molecular weight advantageously promote rapid disintegration of the preparation.

For the preparation according to the invention there is preferred a combination of at least two gel formers; according to a further embodiment, one of the gel formers is insoluble in water.

In a preferred embodiment, a combination of at least one cellulose derivative and a synthetic polymer is preferred for the preparation according to the invention; further preference is given to a combination of at least one water-insoluble cellulose derivative, optionally one or more further cellulose derivatives, and a water-soluble synthetic polymer, and more especially to a combination of water-insoluble ethylcellulose and/or hydroxypropylcellulose and/or hydroxypropylmethyl-cellulose and polyvinylpyrrolidone. For example, in an especially preferred embodiment, for the preparation according to the invention there is preferred a combination of at least two cellulose derivatives, of which at least one is insoluble in water, especially a combination of hydroxypropylcellulose and/or hydroxypropylmethylcellulose and water-insoluble ethyl-cellulose.

The preparation according to the invention can comprise at least one sweetener, flavouring, preservative, colouring and/or filler, preference being given to a content of from 0.1 to 30% by weight, more especially from 1 to 15% by weight, in each case based on the dried preparation.

The preparation according to the invention can have, for example, a film thickness of from 1 to 500 μm, preferably from 1 to 300 μm.

The preparation according to the invention can be in the form of a round, rounded, oval, elliptical, triangular, quadrangular or polygonal film.

Furthermore, the film according to the invention or the preparation according to the invention can be provided with a smooth surface or with a surface having protuberances and/or depressions. Preferably, the surface can have a regular pattern of protuberances and depressions, such as, for example, a wave pattern or a grid pattern.

Furthermore, the film according to the invention or the preparation according to the invention can be provided on a carrier foil.

Furthermore, the film according to the invention or the preparation according to the invention can be provided with a carrier foil made of polyethylene paper (PE paper), polypropylene foil (PP foil) or polyethylene terephthalate foil (PET foil). Preferably, the film according to the invention or the preparation according to the invention is provided on a carrier foil made of polyethylene paper (PE paper), polypropylene foil (PP foil) or polyethylene terephthalate foil (PET foil).

Finally, the film according to the invention or the preparation according to the invention can be provided for oral administration.

Furthermore, an embodiment of the invention relates to a sachet comprising one or more films or preparations according to the invention.

Finally, the invention relates to a multiple-dose container comprising one or more films or preparations according to the invention.

Surprisingly, it has therefore been found that a single-layered film or a single-layered preparation comprising one or more film former(s), one or more gel former(s) and one or more neuroleptic(s), such as, for example, olanzapine, exhibits significantly higher chemical stability than film-coated tablets containing, for example, olanzapine. The film adheres to the oral cavity and disintegrates within a few seconds. For example, the film is dissolved or decomposed by saliva, for example a water-soluble film is dissolved. Accordingly, the film can no longer be spat out. After the film has disintegrated, the active ingredient is mostly swallowed and absorbed in the gastro-intestinal tract. The active ingredient can to some extent be absorbed transmucosally, but this is negligible. The film is preferably substantially free of cavities, surfactants, effervescent additives or taste maskers. The production of the films is substantially more economical than so-called meltable tablets, the production of which requires an elaborate lyophilisation process.

Preferably, the preparation according to the invention comprises at least two film formers. Preferably, the preparation according to the invention comprises at least two gel formers. Special preference is given to a combination of at least two gel formers, one of the gel formers preferably being insoluble in water.

In a preferred embodiment, the preparation according to the invention comprises one or more cellulose derivative(s) and a synthetic polymer, especially a water-insoluble cellulose derivative and a water-soluble synthetic polymer. Preferably, the preparation additionally comprises one or more further film formers, selected from the group consisting of sorbitol, polyethylene glycol, polyethylene glycol dioleate, 1,3-butanediol, propylene glycol, isopropyl palmitate, dibutyl sebacate, xylitol and paraffin oil. Preferably, the preparation additionally comprises one or more further gel formers, especially one or more further cellulose derivatives, more especially one or more cellulose derivatives having a molecular weight of less than 60,000 Dalton, and very especially hydroxypropylcellulose and/or hydroxypropylmethylcellulose.

Such a combination of at least one water-insoluble compound and at least one water-soluble compound has the result that the film-form preparation advantageously releases the active ingredient rapidly and at the same time exhibits sufficiently high stability.

In another preferred embodiment, the preparation according to the invention comprises a plurality of cellulose derivatives, of which one is insoluble in water, especially hydroxypropyl-cellulose and/or hydroxypropylmethylcellulose and water-insoluble ethylcellulose, and one or more compounds selected from the group consisting of sorbitol, polyethylene glycol, polyethylene glycol dioleate, 1,3-butanediol, propylene glycol, isopropyl palmitate, dibutyl sebacate, xylitol and paraffin oil.

Film former:gel former can be present in a ratio of from 0.7:10 to 70:10, preferably from 3:10 to 50:10, especially from 4:10 to 30:10, for example from 5:10 to 15:10. The ratio film former:gel former is very especially from 5:10 to 8:10.

The films can comprise as active ingredient one or more representatives of the group of neuroleptics, e.g. olanzapine, benperidol, haloperidol, clozapine, flupentixol, fluphenazine, droperidol, melperone, flupentixol decanoate, fluspirilene, bromperidol, pimozide, triflupromethazine, risperidone, sertindole, trifluperidol and/or the pharmaceutically acceptable salts thereof. Olanzapine is preferably used as active ingredient.

The active ingredient content in the film can be from 0.1 to 60% by weight and especially up to 50% by weight, preferably 25% by weight, in each case based on the dried preparation.

Furthermore, the film can comprise sweeteners, flavourings, preservatives (e.g. sorbic acid or salts thereof), colourings and/or fillers.

Suitable sweeteners are sucralose, aspartame, cyclamate, saccharine and/or acesulfame, or combinations of those substances.

As flavourings there can be used natural or artificial flavourings, for example lemon, orange, strawberry, vanilla or peppermint flavouring, cinnamyl acetate, citral, citronella, eugenyl formate, menthol and/or methylanisole.

As colourings there can be used pharmaceutically customary flavourings and pigments, especially TiO₂, FeₓOₓ, β-carotene, azorubin, indigotin, riboflavin and the like.

As fillers there can be used salts, such as carbonates, phosphates, oxides, such as e.g. SiO₂, especially in the form of Aerosil, or the like and/or cellulose and derivatives thereof, and also sparingly soluble sugars and sugar derivatives, such as, for example, lactose or starch derivatives such as cyclodextrins, provided they are in substantially undissolved form in the product and therefore fulfil the mechanical properties of a filler. Preferably, SiO₂ is used as filler.

The thickness of the film can be from 1 to 500 μm, preferably from 1 to 300 μm. In order to avoid an unpleasant sensation in the mouth, the film thickness must not be too great.

The films can have round, oval, elliptical, triangular, quadrangular or polygonal shapes; they can, however, also have any rounded shape.

The surface of the films can be smooth or provided with protuberances or depressions.

The disintegration time of the films in the oral cavity is less than 200 seconds, preferably from 10 to 60 seconds, especially from 10 to 30 seconds.

For the preparation of the film, the active ingredient(s) is(are) suspended or dissolved in a solvent. Alcohols or alcohol/water mixtures can be used as solvent. After the addition of film formers, gel formers and optionally sweeteners, flavourings, colourings and/or fillers, the mixture is homogenised. The mixture is applied to a carrier material with the aid of a suitable coating method. As carrier material there can be used, for example, PE paper or PP or PET foil. The coated carrier material is dried at from 30 to 120° C., preferably at from 30 to 70° C. The coated carrier material is then processed further to form separate films of defined area. This can be effected by punching, cutting or stamping. The films are individually packed into sachets with or without carrier foil. They can also be packed into multiple-dose containers. Prior to administration, where applicable the active-ingredient-containing film is removed from the carrier material.

The film-form preparation is used according to the invention for the administration of neuroleptics in the treatment of a disorder in the central nervous system, the treatment of schizophrenia, the treatment of a schizophreniform disease, the treatment of acute mania, the treatment of mild anxiety states and the like. Preferably, the film-form preparation is used to produce a medicament for the treatment of a disorder in the central nervous system, the treatment of schizophrenia, the treatment of a schizophreniform disease, the treatment of acute mania, the treatment of mild anxiety states and the like.

The invention is explained in greater detail by the following Examples, but without thereby limiting the scope of the invention.

Unless otherwise indicated, all percentages given in % by weight relate to the dried preparation.

### EXAMPLE 1

The following substances are used for producing olanzapine films.

| Constituents | Percent (%) | Weight (g/100 g) |
|---|---|---|
| Olanzapine | 50 | 50 |
| Hydroxypropylmethylcellulose | 30 | 30 |
| Ethylcellulose | 5 | 5 |
| Paraffin oil | 5 | 5 |
| D-Sorbitol | 5 | 5 |
| 1,3-Butanediol | 2.5 | 2.5 |
| Isopropyl palmitate | 2.5 | 2.5 |
| Ethanol/water | | 240* |

*is removed during the preparation process

### Preparation:

For the preparation of the film, first of all the D-sorbitol is dissolved in water. 1,3-Butanediol, isopropyl palmitate, paraffin oil and ethanol as solvent are added to the resulting solution and the mixture is stirred. Then first the ethyl-cellulose and the hydroxypropylmethylcellulose are added and dissolved and subsequently the olanzapine is weighed in and the resulting suspension is homogenised using a suitable stirring device.

The mixture is subsequently spread out on a suitable carrier, for example PE foil, using a coating machine and the ethanol/water mixture is removed at 50° C. The film so obtained is then punched out in accordance with the dosage and packaged.

Comparison of the Stability of the Olanzapine Film with a Film-Coated Olanzapine Tablet

| | | | | Olanzapine film- |
|---|---|---|---|---|
| Storage | | Storage | Olanzapine film | coated tablet |
| period | | conditions | Impurities | Impurities |
| 0 | months | not monitored | 0.03 | 0.37 |
| 0.5 | month | 40° C./75% | 0.03 | |
| | | rel. humidity | | |
| 3 | months | 25° C./60% | | 0.43 |
| | | rel. humidity | | |
| 3 | months | 40° C./75% | | 0.73 |
| | | rel. humidity | | |

As can be seen from the above Table, appreciable amounts of impurities can be detected in olanzapine-containing film-coated tablets, in some cases even shortly after production, which amounts increase on further storage. In comparison therewith, barely detectable impurities are formed in the film preparation.

### EXAMPLE 2

| Constituents | Percentage (%) | Weight (g/100 g) |
|---|---|---|
| Olanzapine | 25 | 25 |
| Hydroxypropylcellulose | 15 | 15 |
| Polyvinylpyrrolidone | 37 | 37 |
| D-Sorbitol | 10 | 10 |
| 1,3-Butanediol | 8 | 8 |
| Ethylcellulose | 5 | 5 |
| Ethanol/water | | 240* |

*is removed during the preparation process

Preparation is carried out analogously to Example 1.

### EXAMPLE 3

Analogously to Example 1, films of the composition shown in the following Table containing diffent dosages of olanzapine were prepared.

| | Amount in mg | |
|---|---|---|
| Constituent | per film | Amount in % |
| Olanzapine | 5, 10, 15, 20 | 25 |
| Hydroxypropylmethylcellulose | | 15 |
| Ethylcellulose | | 5 |
| Sorbitol | | 8.5 |
| Dibutyl sebacate | | 5 |
| Isopropyl palmitate | | 3.5 |
| PEG | | 2 |
| Polyvinylpyrrolidone | | 25 |
| Aerosil | | 9 |
| Sucralose | | 1.5 |
| Orange flavouring | | 0.5 |

In blood level curves, the film preparations so prepared exhibited active ingredient blood levels comparable with those of film-coated tablets each of the same dosage.

1-30. (canceled)

31. A film-form preparation comprising one or more film former(s), one or more gel former(s) and one or more active ingredient(s) from the group of neuroleptics.

32. The film-form preparation of claim 31, wherein the preparation is a solid film.

33. The film-form preparation of claim 31, wherein the preparation is single-layered.

34. The film-form preparation of claim 31, wherein the preparation is free of cavities.

35. The film-form preparation of claim 31, wherein the preparation is free of surfactants.

36. The film-form preparation of claim 31, wherein the preparation is free of effervescent additive.

37. The film-form preparation of claim 31, wherein the preparation is free of taste maskers.

38. The film-form preparation of claim 31, wherein the neuroleptic is selected from the group consisting of olanzapine, benperidol, haloperidol, clozapine, flupentixol, fluphenazine, droperidol, melperone, flupentixol decanoate, fluspirilene, bromperidol, pimozide, triflupromethazine, risperidone, sertindole, and trifluperidol and pharmaceutically acceptable salts thereof.

39. The film-form preparation of claim 38, wherein the neuroleptic is olanzapine.

40. The film-form preparation of claim 31, wherein the active ingredient content in the preparation is from 0.1% to 60% by weight.

41. The film-form preparation of claim 31, wherein the active ingredient content in the preparation is up to 50% by weight.

42. The film-form preparation of claim 31, wherein the active ingredient content in the preparation is from 20% to 30% by weight.

43. The film-form preparation of claim 31, wherein the active ingredient content in the preparation is about 25% by weight.

44. The film-form preparation of claim 31, wherein the film former is a sugar or sugar alcohol, or derivative thereof; a low molecular weight organic acid; polyethylene glycol, polyethylene glycol dioleate, 1,3-butanediol, propylene glycol, glycerol, isopropyl palmitate, dibutyl sebacate, paraffin oil or castor oil; ethylcellulose; cellulose acetate; cellulose phthalate and a mixture thereof.

45. The film-form preparation of claim 44, wherein the sugar is saccharose, sorbitol, mannitol, xylitol, glucose, fructose, lactose or galactose.

46. The film-form preparation of claim 44, wherein the low molecular weight organic acid is citric acid, succinic acid, malic acid or adipic acid.

47. The film-form preparation of claim 44, wherein film former is a mixture of ethylcellulose, cellulose acetate, cellulose phthalate, sorbitol, xylitol, polyethylene glycol, 1,3-butanediol, propylene glycol, isopropyl palmitate, dibutyl sebacate and paraffin oil.

48. The film-form preparation of claim 31, wherein the film former is in an amount of from 5% to 70% by weight.

49. The film-form preparation of claim 31, wherein the gel former is a polymeric carbohydrate; a synthetic polymer that is soluble or swellable in water; a polypeptide or a mixture thereof.

50. The film-form preparation of claim 49, wherein the polymeric carbohydrate is cellulose, a cellulose derivative, starch, a starch derivative, agar-agar, alginic acid, arabinogalactan, galactomannan, carrageenan, dextran, tragacanth or gum of vegetable origin.

51. The film-form preparation of claim 50, wherein the cellulose derivative is hydroxypropylmethylcellulose or hydroxypropylcellulose.

52. The film-form preparation of claim 49, wherein the synthetic polymer is polyvinylpyrrolidone, polyvinyl alcohol, polyacrylic acid or polyacrylamide.

53. The film-form preparation of claim 49, wherein the polypeptide is gelatin, albumin or collagen.

54. The film-form preparation of claim 50, wherein the cellulose derivative has a molecular weight of less than 60,000 Dalton.

55. The film-form preparation of claim 31, wherein the gel former is in an amount of from 10% to 70% by weight.

56. The film-form preparation of claim 31, further comprising a sweetener, a flavoring, a preservative, a coloring or a filler.

57. The film-form preparation of claim 31, wherein the film-form preparation has a thickness of from 1 μm to 500 μm.

58. The film-form preparation of claim 31, wherein the shape of the film-form preparation is round, rounded, oval, elliptical, triangular, quadrangular or polygonal.

59. The film-form preparation of claim 31, wherein the film-form preparation has a smooth surface or a surface having protuberances or depressions.

60. The film-form preparation of claim 31, wherein the film-form preparation is arranged on a carrier foil.

61. The film-form preparation of claim 60, wherein the carrier foil is polyethylene paper, polypropylene foil or polyethylene terephthalate foil.

62. The film-form preparation of claim 31, wherein the film-form preparation is for oral administration.

63. A sachet comprising one or more of the film-form preparations of claim 31.

64. A multiple-dose container comprising one or more of the film-form preparations of claim 31.

65. A method for producing the film-form preparation of claim 31 comprising
(a) dissolving the film former in a suitable solvent;
(b) adding the gel former to the dissolved film former;
(c) adding the active ingredient to the mixture of step (b);
(d) homogenizing the mixture of step (c);
(e) applying the homogenized mixture to a suitable carrier; and
(f) removing the solvent thereby producing a film-form preparation.

66. A method for treating a disorder of the central nervous system comprising administering a film-form preparation of claim 31 to a subject with schizophrenia, a schizophreniform disease, acute mania, or a mild anxiety state thereby treating the disorder of the central nervous system.
